(19) United States
(12) Patent Application Publication
(10) Pub. No.: US 2023/0086506 A1
(43) Pub. Date: Mar. 23, 2023

(54) FLUIDIC DEVICE FOR MODULAR TISSUE ENGINEERING AND METHODS OF USE

(71) Applicant: UNIVERSITY OF MIAMI, Miami, FL (US)

(72) Inventors: Ashutosh Agarwal, Miami Beach, FL (US); Vera Mayo, Miami, FL (US); Matthew Ishahak, Miami, FL (US)

(21) Appl. No.: 17/909,303

(22) PCT Filed: Mar. 5, 2021
(86) PCT No.: PCT/US21/21179

§ 371 Date: Sep. 2, 2022

Related U.S. Application Data
(60) Provisional application No. 62/986,007, filed on Mar. 6, 2020.

(51) Int. Cl. C12M 3/00; C12M 1/42; C12M 3/06; C12M 1/12

(52) U.S. Cl. CPC C12M 21/08; C12M 35/08; C12M 23/16; C12M 25/06

(57) ABSTRACT

A microfluidic structure includes a first media channel or well and a second media channel. A removable membrane is provided between the first media channel or well and the second media channel to permit diffusion. One or more plates is used to form the second media channel. A method of creating a microenvironment using the microfluidic structure is also provided.

### CROSS-REFERENCE TO RELATED APPLICATION

Priority is claimed to U.S. Provisional Patent Application No. 62/986,007, filed Mar. 6, 2020, the entire disclosure of which is incorporated herein by reference.

### STATEMENT OF GOVERNMENT SUPPORT

This invention was made with government support under 1UC4DK104208-01 and F31DK118860 awarded by the National Institutes of Health. The Government has certain rights in the invention.

### FIELD OF THE INVENTION

The present disclosure relates to a fluidic device for modular tissue engineering and, more particularly, to a fluidic device with a removable cell culture membrane permitting diffusion between a first media and a second media.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Cellular extravasation and mass transport across cellular barriers are central events of human physiology. For example, breast cancer circulating tumor cells establish metastatic tumors in the bone due to a close interaction with local cells including endothelial cells, perivascular mesenchymal stem cells (hMSC), and osteoblasts. Other cellular microenvironments, in which other kinds of cells interact, are also known to be involved in the development of various diseases such as cancer. In vitro recapitulation of microenvironments could provide new insight into, for example, the metastatic cascade, and could aid in developing new therapeutic strategies.

Microfluidic platforms designed with fluidic channel architectures offer the prospect of accurately simulating cancer-supporting microenvironments as well as biologic barrier functions. However, more development is needed to accurately replicate such environments and to permit retrieval and testing of cells.

### SUMMARY OF THE INVENTION

The present techniques provide microplate platform structures and microfluidic channel structures for modular tissue engineering. The structures provide for both manual seeding and flowing in of cells to create an organ chip model for research. Further, the structures allow cells to be retrieved for testing. The present techniques include improvements to methods of testing various pathologies. For example, a device may be fabricated to include key components of the bone marrow niche: osteoblasts and a vascular wall. Such a device is compatible with cancer cell culture, including upregulation of metastatic markers and can serve as an extravasation model by being optimized to promote migration of cancer cells through the engineered vascular membrane. Other cell types besides osteoblasts are considered within the scope of the disclosure.

In accordance with an example, an apparatus includes a first plate, a second plate, a first media channel, and a second media channel. The first plate has an upper surface, a lower surface, a proximal end, and a distal end. The second plate has an upper surface, a proximal end, and a distal end. The upper surface of the second plate configured for placement adjacent the lower surface of the first plate. The first media channel is disposed in the first plate and has a flow direction from the proximal end to the distal end of the first plate. The first media channel includes a first media inlet, an upper compartment, a removable membrane, and a first media outlet. The first media inlet is in the upper surface of the first plate. The upper compartment is downstream of the first media inlet in the flow direction. The removable membrane is downstream of the first media inlet in the flow direction and provided in a passageway that extends through the lower surface of the first plate. The first media outlet is in the upper surface of the first plate downstream of the removable membrane in the flow direction. The second media channel includes a second media inlet, a lower compartment, and a second media outlet. The second media inlet is in the upper surface of the second plate. The lower compartment extends through the upper surface of the second plate downstream of the second media inlet in the flow direction and is configured for placement adjacent the passageway of the removable membrane of the first media channel. The second media outlet is in the upper surface of the second plate downstream of the lower compartment.

In accordance with another example, an apparatus includes a plate having an upper surface, a lower surface, a proximal end, a distal end, and an intermediate plane. A first media channel is disposed in the plate at or above the intermediate plane. The first media channel has a flow direction from the proximal end to the distal end of the plate. The first media channel includes a first media inlet, a removable membrane, and a first media outlet. The first media inlet is in the upper surface of the first plate. The removable membrane is downstream of the first media inlet in the flow direction and is provided in a passageway extending through the intermediate plane. A first media outlet is in the upper surface of the plate downstream of the removable membrane in the flow direction. A second media channel is disposed in the plate. The second media channel has a flow direction from the proximal end to the distal end of the plate. The second media channel includes a second media inlet, a lower pathway, a compartment, and a second media outlet. The second media inlet is in the upper surface of the plate. The lower pathway is disposed below the intermediate plane downstream of the second media inlet. The compartment is fluidly connected to the lower pathway and is further connected to the passageway of the removable membrane of the first media channel. The second media outlet is in the upper surface of the plate downstream of the compartment.

In accordance with another example, an apparatus includes a well, a first plate, a removable membrane, a second plate, and a second media channel. The well has a first media receptacle and an open bottom. The first plate has an upper surface, a lower surface, a proximal end, and a distal end. The upper surface of the first plate is configured for placement adjacent the open bottom the well. The lower surface of the first plate includes a recessed trench. The removable membrane is configured for placement in a passageway between the open bottom of the well and the upper surface of the first plate. The second plate has an upper surface, a proximal end, and a distal end. The upper surface of the second plate is configured for placement adjacent the lower surface of the first plate with the proximal ends and distal ends of the first plate and the second plate aligned. The upper surface of the second plate includes a raised trough. The second media channel is formed between the recessed trench of the first plate and the raised trough of the second plate. The second media channel has a flow direction from the proximal ends of the first plate and the second plate to the distal ends of the first plate and the second plate. The second media channel includes a second media inlet aperture, a compartment, and a second media outlet. The second media inlet aperture is through the first plate. The compartment is through the first plate downstream of the second media inlet aperture in the flow direction and is configured for placement adjacent the passageway of the removable membrane. The second media outlet is through the first plate.

In some forms, the apparatus may have two first media channels and two second media channels. In other forms, the apparatus may have two wells, two removable membranes, two passageways, and two second media channels, each well having a first media receptacle.

In some forms, the first plate further may include a second media inlet aperture and a second media outlet aperture. The second media inlet aperture may be configured for placement adjacent the second media inlet of the second plate. The second media outlet aperture may be configured for placement adjacent the second media outlet of the second plate.

In some forms, the apparatus may further include a cap having a lower surface, a first media inlet aperture, and a first media outlet aperture. The lower surface of the cap may be configured for placement adjacent the upper surface of the first plate. The first media inlet aperture may be configured for placement adjacent the first media inlet of the first plate. The first media outlet aperture may be configured for placement adjacent the first media outlet of the first plate. The lower surface of the cap may cover the first media channel from the first media inlet to the first media outlet. The plate may include a detent, and the cap may include an extension that is complementary to the detent and configured for placement in the detent.

In some forms, the apparatus may further include a cap having a lower surface, a first media inlet aperture, a first media outlet aperture, a second media inlet aperture, and a second media outlet aperture. The lower surface of the cap may be configured for placement adjacent the upper surface of the plate. The first media inlet aperture may be configured for placement adjacent the first media inlet. The first media outlet aperture may be configured for placement adjacent the first media outlet. The second media inlet aperture may be configured for placement adjacent the second media inlet. The second media outlet aperture may be configured for placement adjacent the second media outlet.

In some forms, the upper compartment may be populated with a first type of cells.

In some forms, the removable membrane may include a second type of cells on a first side and a third type of cells on a second side.

In some forms, a first media may be circulated through the first media channel. In other forms, a first media may be contained in the first media receptacle. A second media may be circulated through the second media channel. The first media may include media conditioned by a first type of cells and the second media may include a fourth type of cells. The removable membrane may be exposed to the first media and may allow cytokine diffusion through the removable membrane and into the second media in the second media channel.

In accordance with yet another example, a method of creating a microenvironment includes supplying an apparatus including a first media location, a second media channel, and a removable membrane between the first media location and the second media channel. The method includes populating the removable membrane with a first kind of cells and a second kind of cells. The method further includes providing a first media in the first media location, flowing a second media having a third kind of cells through the second media channel, and allowing diffusion through the removable membrane.

In some forms, the method may further include culturing at least one of the first kind of cells, the second kind of cells, and the third kind of cells over a period of time.

In some forms, the second media may flow through the second media channel at a flow rate between five microliters per minute and one milliliter per minute.

In some forms, the first media location may be a first media channel. Providing a first media in the first media location may include flowing the first media through the first media channel. In other forms, the first media location may be a well having a first media receptacle and an open bottom.

### BRIEF DESCRIPTION OF THE DRAWINGS

The figures described herein depict various aspects of the system and methods disclosed herein. It should be understood that each figure depicts an example of aspects of the present systems and methods.

FIG. 1A illustrates an exploded view of a microfluidic channel assembly, in an example, having two first media channels disposed in a first plate and two second media channels disposed in a second plate and a cap.

FIG. 1B illustrates a cross-sectional side view of the microfluidic channel assembly shown in FIG. 1A.

FIG. 2A illustrates an exploded view of a microfluidic channel assembly, in an example, having one first media channel disposed in a first plate and one second media channel disposed in a second plate.

FIG. 2B illustrates a cross-sectional side view of the microfluidic channel assembly shown in FIG. 2A.

FIG. 3A illustrates a cross-sectional side view of a microfluidic channel assembly, in an example, having a well and a second media channel formed between a first plate and a second plate.

FIG. 3B illustrates an isometric view of an upper surface of the second plate of the microfluidic channel assembly shown in FIG. 3A.

FIG. 3C illustrates an isometric view of a lower surface of the first plate of the microfluidic channel assembly shown in FIGS. 3A and 3B.

FIG. 3D illustrates an isometric view of the assembled first plate and second plate of the microfluidic channel assembly shown in FIGS. 3A-3C.

FIG. 4 illustrates schematically a method of creating a microenvironment.

### DETAILED DESCRIPTION

A newly designed set of fabricated microstructures are provided that may be used as organ chip models for research. The apparatuses, as described, are useful for creating a variety of microenvironments that are optimized for research purposes, including but not limited to a bone microenvironment. A method of creating a microenvironment is also provided herein.

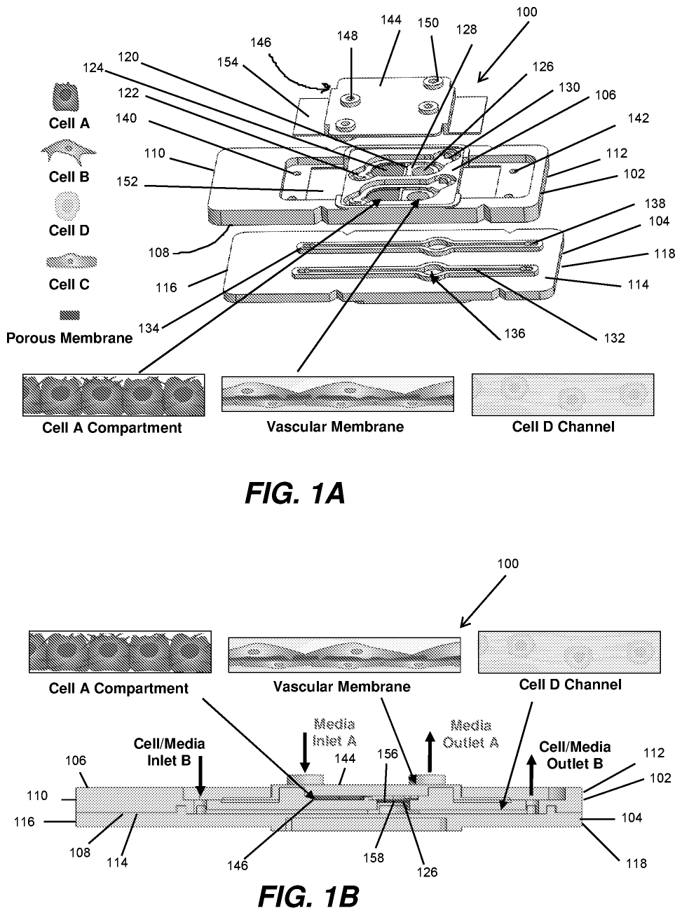

FIG. 1A illustrates a first example of a microfluidic channel assembly 100 in an exploded view. The microfluidic channel assembly 100 includes a first plate 102 and a second plate 104. The first plate 102 has an upper surface 106, a lower surface 108 (better shown in FIG. 1B), a proximal end 110, and a distal end 112. Similarly, the second plate 104 has an upper surface 114, a proximal end 116, and a distal end 118. For purposes of this specification, the upper surface 106, lower surface 108, and upper surface 114 are all understood to refer to respective planes of the first plate 102 or second plate 104 that include an exterior wall of the first plate 102 or second plate 104 and may also include open spaces, apertures, or portions of raised or lowered sections where other features of the first plate 102 or second plate 104 are provided in or extend through the upper surface 106, lower surface 108, or upper surface 114. When the microfluidic channel assembly 100 is assembled, the upper surface 114 of the second plate 104 is adjacent the lower surface 108 of the first plate. Further, the respective proximal ends 110 and 116 and the respective distal ends 110 and 118 of the first plate 102 and the second plate 104 are aligned.

As shown in FIG. 1A, a first media channel 120 is disposed in the first plate 102. The first media channel 120 has a flow direction from the proximal end 110 to the distal end 112 of the first plate 102. The first media channel 120 includes a first media inlet 122 in the upper surface of the first plate 102, an upper compartment 124 downstream of the first media inlet 122 in the flow direction, a removable membrane 126 downstream of the first media inlet 122 in the flow direction, and a first media outlet 130 downstream of the removable membrane 126 in the flow direction. In the arrangement shown, the first media channel 120 is open through the upper surface 106 of the first plate 102.

In the first media channel 120 shown in FIG. 1A, the removable membrane 126 is provided in a passageway 128 that extends through the lower surface 108 of the first plate 102. The removable membrane 126 is porous and can be any commonly used cell culture membrane. The removable membrane 126 is removable in order to allow cell retrieval. As shown in FIG. 1A, the removable membrane 126 is downstream of the upper compartment 124 and has a circular shape. The dimensions of the removable membrane 126 can be adjusted depending on the specific organ application for which the microfluidic channel assembly 100 is to be used. Likewise, the dimensions of the first media channel 120 can be adjusted depending on the specific organ application for which the microfluidic channel assembly 100 is to be used. The microfluidic channel assembly 100 may have a first media channel 120 with a width between 50 μm and 1 cm.

As further shown in FIG. 1A, a second media channel 132 is disposed in the second plate 104. The second media channel 132 has a flow direction from the proximal end 116 to the distal end 118 of the second plate 104. The second media channel 132 includes a second media inlet 134 in the upper surface 106, a lower compartment 136 downstream of the second media inlet 134, and a second media outlet 138 in the upper surface 114 of the second plate 104 downstream of the lower compartment 136. The lower compartment 136 extends through the upper surface 114 of the second plate 104. When the microfluidic channel assembly 100 is assembled, the lower compartment 136 is adjacent the passageway 128 of the removable membrane 126.

In the arrangement shown in FIG. 1A, the microfluidic channel assembly has two first media channels 120 and two second media channels 132. Each first media channel 120 is connected via a passageway 128 to one second media channel 132. In the arrangement shown, the first media channels 120 are substantially parallel and are substantially aligned. In other arrangements, the first media channels 120 may be disposed at an angle relative to one another and/or may be staggered or offset from one another. In the arrangement shown, the second media channels 132 are substantially parallel and are substantially aligned. In other arrangements, the second media channels 132 may be disposed at an angle relative to one another and/or may be staggered or offset from one another. A gasket or gaskets may be provided between the first plate 102 and second plate 104 to ensure a sealed engagement between each first media channel 120 and each second media channel 132. The gasket or gaskets may be compressible and/or elastomeric.

As also shown in FIG. 1A, the first plate 102 includes a second media inlet aperture 140 and a second media outlet aperture 142. When the microfluidic channel assembly 100 is assembled, the second media inlet aperture 140 is adjacent the second media inlet 134 of the second plate 106 and the second media outlet aperture 142 is adjacent the second media outlet 138. The enables a second media to flow through the second media inlet aperture 140 in the first plate 102, into the second media channel 132 in the second plate 104 via the second media inlet 134, out of the second media channel 132 in the second plate 104 via the second media outlet 138, and through the second media outlet aperture 142 in the first plate 102.

Referring still to FIG. 1A, a cap 144 may be provided to cover the first plate 102. The cap 144 may have a lower surface 146 (better shown in FIG. 1B), a first media inlet aperture 148, and a first media outlet aperture 150. For purposes of this specification, the lower surface 146 is understood to refer to a plane of the cap 144 that includes an exterior wall of the cap 144 and may also include open spaces or apertures or portions of raised or lowered sections of other features of the microfluidic channel assembly 100 that are provided in or extend through the lower surface 146. As shown in FIG. 1A, when the microfluidic channel assembly 100 is assembled, the lower surface 146 of the cap 144 is adjacent the upper surface 106 of the first plate 102. A first media is able to flow through the first media inlet aperture 148 in the cap 144 into the first media channel 120 in the first plate 102 via the first media inlet 122. The first media can then flow out of the first media channel 120 via the first media outlet 130 and through the first media outlet aperture 150 in the cap 144. When the first media channel 120 is open through the upper surface 106 of the first plate 102, the cap 144 covers the first media channel 120 from the first media inlet 122 to the first media outlet 130. The cap 144 thus serves as a top of the first media channel 120, including as a top of the upper compartment 124 and passageway 128. The first plate 102 includes a detent 152 of first plate 102, and the cap 144 includes an extension 154 that is complementary to the detent 152. When the microfluidic channel assembly 100 is assembled, the extension 154 of the cap is secured to the detent 152 of the first plate. Securement options include clamps, adhesives, fasteners, and a press fit, as well as any other common securement or fastening methods. Such securement options may alternately or in addition be used elsewhere on the first plate 102 or cap 144. A gasket or gaskets may be provided between the first plate 102 and the cap 144 to ensure a sealed engagement. The gasket or gaskets may be compressible and/or elastomeric.

FIG. 1B shows the microfluidic channel assembly 100 in use. The upper compartment 124 is populated with a first type of cells, identified as Cells A, such as osteoblasts. The removable membrane 126 is also populated with cells. For example, a second type of cells, identified as Cells C, such as endothelial cells may be provided on a first side 156 of the removable membrane 126 and a third type of cells, identified as Cells B, such as perivascular cells, may be provided on a second side 158 of the removable membrane 126. A first media is circulated through the first media channel 120 (shown in FIG. 1A). As described herein, the term media may refer to a media or a conditioned media. The first media may, for example, include media conditioned by the first type of cells (Cells A), such as osteoblast-conditioned media. A second media is circulated through the second media channel 132 (shown in FIG. 1A). The second media may include a fourth type of cells, identified as Cells D. For example, the second media may be circulating tumor cells (CTC) media. The removable membrane 126 is exposed to the first media and allows diffusion from the first media channel 120 to the second media channel 132. For example, when the first media includes osteoblast-conditioned media and the second media includes circulating tumor cells (CTC) media, the removable membrane 126 allows cytokine diffusion from the first media channel 120 to the second media channel 132.

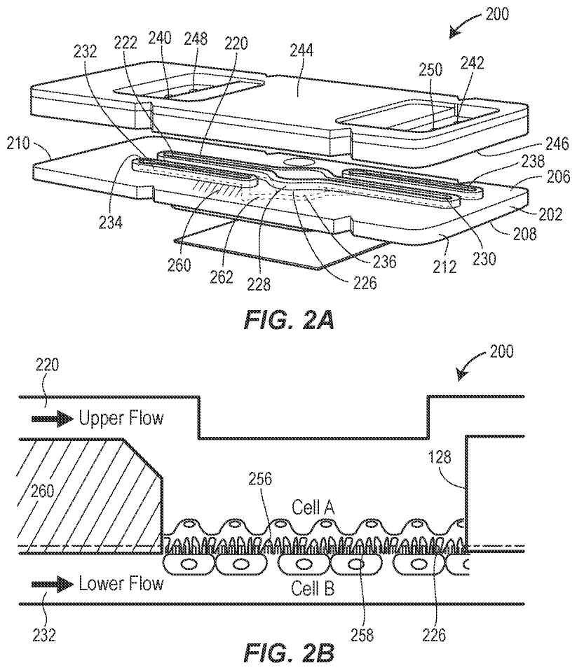

FIG. 2A shows an exploded view of a microfluidic channel assembly 200 that is a variation of the microfluidic channel assembly 100 shown in FIGS. 1A and 1B. The microfluidic channel assembly 200 operates generally in the same manner as the microfluidic channel assembly 100 with minor structural differences. Instead of a first plate 102 and a second plate 104, a single plate 202 includes both the first media channel 220 and the second media channel 232. As with first plate 102, the plate 202 has an upper surface 206, a lower surface 208, a proximal end 210, and a distal end 212. The plate 202 further includes an intermediate plane 260. The first media channel 220 is disposed in the plate 202 at or above the intermediate plane 260. The second media channel 232 includes a lower pathway 262 below the intermediate plane 260 that is fluidly connected to a compartment 236. The passageway 128 of the removable membrane 226 of the first media channel 220 extends through the intermediate plane 260 and is connected to the compartment 236. That permits, for example, diffusion between the first media channel 220 and the second media channel 232. A first media inlet 222, a first media outlet 230, a second media inlet 234, and a second media outlet 230 are all provided in the upper surface 206 of the plate 202. Although the plate 202 does not include an upper compartment 224 (such as upper compartment 124) in the arrangement shown in FIG. 2A, alternate arrangements of the plate 202 could include an upper compartment 224 that could, for example, be populated with osteoblasts.

As further shown in FIG. 2A, the microfluidic channel assembly 200 includes a cap 244. The cap 244 has a lower surface 246 that, when the microfluidic channel assembly 200 is assembled, is located adjacent the upper surface 206 of the plate 202. The lower surface 246 of the cap 244 covers the first media channel 220 from the first media inlet 222 to the first media outlet aperture 230. The second media channel 232 may also be covered, in whole or in part, from a second media inlet 234 to a second media outlet 238. A first media inlet aperture 248, a first media outlet aperture 250, a second media inlet aperture 240, and a second media outlet aperture 242 are provided in the cap 244 and are placed adjacent, respectively, the first media inlet 222, the first media outlet 230, the second media inlet 234, and the second media outlet 238 of the plate 202 when the microfluidic channel assembly 200 is assembled.

FIG. 2B illustrates the tiered flow of a first media and a second media through the microfluidic channel assembly 200. The upper flow is shown in the first media channel 220, and the lower flow is shown in the second media channel 232. The removable membrane 226 is located between the first media channel 220 and the second media channel 232 in passageway 228. As previously discussed with respect to FIG. 1B, the removable membrane 226 may be populated on a first side 256 with one type of cells, such as endothelial cells, and on a second side 258 with another type of cells, such as perivascular cells. The removable membrane 226 is exposed to the first media and allows diffusion from the first media channel 220 to the second media channel 232. For example, when the first media includes osteoblast-conditioned media and the second media includes circulating tumor cells (CTC) media, the removable membrane 226 allows cytokine diffusion from the first media channel 220 to the second media channel 232. Although the arrangement depicted in FIG. 2B shows only two tiers of flow, alternate arrangements are contemplated that include additional tiers of flow (e.g., a third media channel below the second media channel, a fourth media channel below the third media channel, etc.) that could be connected by removable membranes populated with identical or different cell populations.

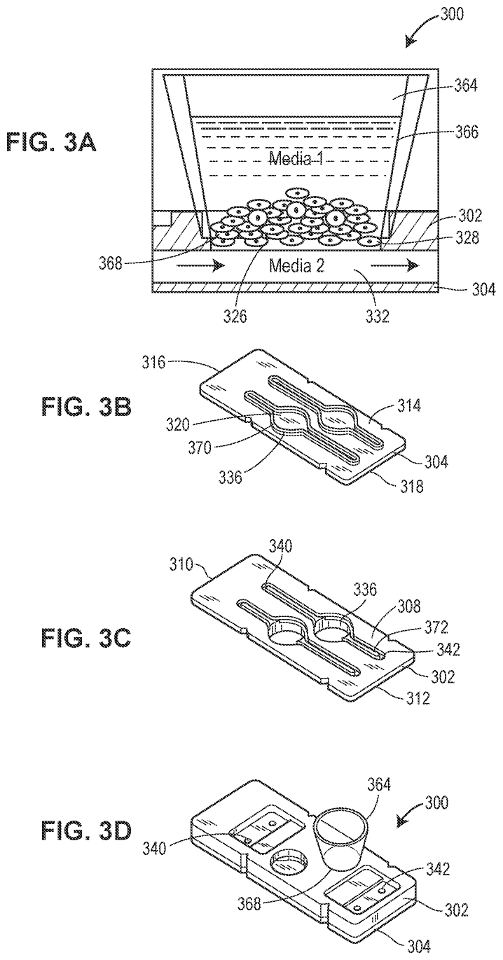

FIG. 3A illustrates a side view of a microfluidic channel assembly 300 that is another variation of the microfluidic channel assemblies 100 and 200 shown in FIGS. 1A-2B. The microfluidic channel assembly 300 operates in generally the same manner as the microfluidic channel assemblies 100 and 200 except, instead of a first media channel 120 or 220 through which a first media flows, the microfluidic channel assembly 300 has a well 364 having a first media receptacle 366 in which the first media is kept. In addition, the second media channel 332 is formed between a first plate 302 and a second plate 304 instead of being disposed in a single plate

More particularly, as shown in FIG. 3A, the well 364 has an open bottom 368. When the microfluidic channel assembly 300 is assembled, the passageway 328 of the removable membrane 326 is located between the open bottom 368 of the well 364 and an upper surface 306 of the first plate 302 and the removable membrane 326 is placed in the passageway 328. A first media is kept in the well 364, and a second media flows through the second media channel 332 below the well. The removable membrane 326 is thus exposed to the first media and allows diffusion from the well 364 to the second media channel 332.

FIG. 3B shows the upper surface 314 of the second plate 304. The second plate 304 has a proximal end 316 and a distal end 318. When the microfluidic channel assembly 300 is assembled, the upper surface 314 of the second plate is configured for placement adjacent a lower surface 308 of the first plate 302 (shown in FIG. 3C). Returning to FIG. 3B, the second plate 304 has a raised trough 370 that forms in part the second media channel 320. The raised trough 370 includes an expanded area that forms in part a compartment 336.

FIG. 3C shows the lower surface 308 of the first plate 302 (i.e., the first plate 302 is shown upside down from its assembled orientation). The first plate 302 has a proximal end 310 and a distal end 312. When the microfluidic channel assembly 300 is assembled, the proximal ends 310 and 316 and the distal ends 312 and 318 of the first plate 302 and the second plate 304, respectively, are aligned. The lower surface 308 includes a recessed trench 372 that, in conjunction with the raised trough 370, forms the second media channel 332. The raised recessed trench 372 has a substantially identical outer boundary as the raised trough 370, including an expanded area that forms in part the compartment 336. A gasket or gaskets may be provided between, along, or around the recessed trench 372 and the raised trough 370 to maintain a seal of the second media channel 332. The second media channel 332 has a flow direction from the aligned proximal ends 310 and 316 of the first plate 302 and the second plate 304 to the aligned proximal ends 312 and 318 of the first plate 302 and the second plate 304. As shown in FIGS. 3C and 3D, the first plate 302 has a second media inlet aperture 340 and a second media outlet aperture 342 to permit a second media to travel into and out of the second media channel 332 through the first plate 302.

FIG. 3D shows the microfluidic channel assembly 300 as assembled. The upper surface 306 of the first plate 302 is placed adjacent the open bottom 368 of the well 364. As better shown in FIG. 3A, the compartment 336 is adjacent the passageway 328 containing the removable membrane 326. As shown in FIG. 3D, the microfluidic channel assembly 300 can accommodate two wells 364 (though only one is shown), each having a first media receptacle 366. Accordingly, the microfluidic channel assembly 300 further may include two removable membranes 326, two passageways 328, and two second media channels 332. In other arrangements not herein shown, the microfluidic channel assembly 300 may have additional wells 364 and second media channels 332 running in parallel. Additional parallel channels are also possible with microfluidic channel assemblies 100 and 200.

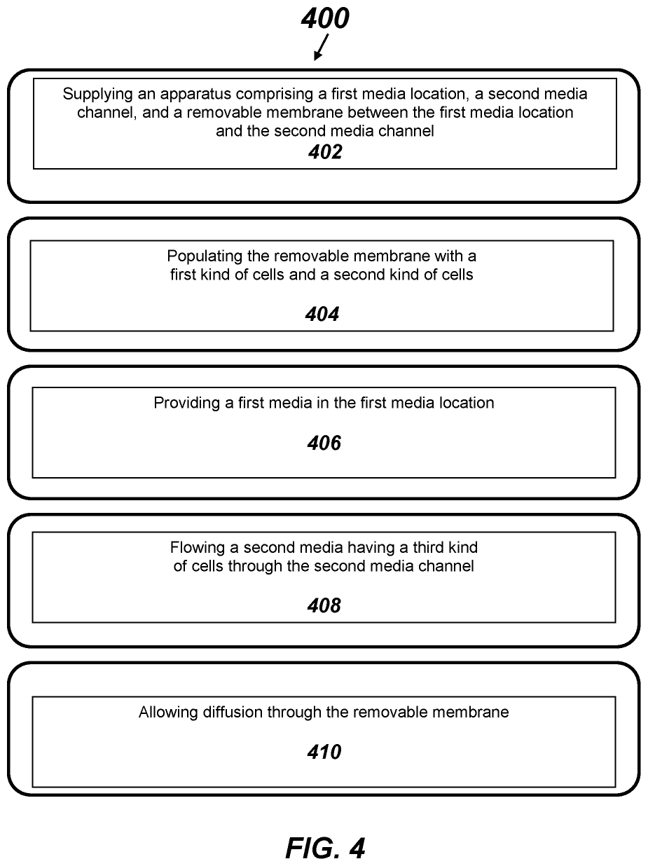

FIG. 4 shows a schematic representation of a method 400 of using any of the microfluidic channel assemblies described herein. At box 402, the method 400 includes supplying an apparatus comprising a first media location, a second media channel, and a removable membrane between the first media location and the second media channel. The first media location may be either a first media channel (as described with respect to microfluidic channel assemblies 100 and 200) or a well having a first media receptacle and an open bottom (as described with respect to microfluidic channel assembly 300). At box 404, the method 400 includes populating the removable membrane with a first kind of cells and a second kind of cells. As a non-limiting example, the first kind of cells may be endothelial cells and the second kind of cells may be perivascular cells. At box 406, the method 400 includes providing a first media in the first media location. Providing a first media in the first media location may include flowing the first media through a first media channel. At box 408, the method 400 includes flowing a second media having a third kind of cells through the second media channel. The third kind of cells may be circulating tumor cells (CTCs), for example. Other kinds of cells could alternately be used. The second media may flow through the second media channel at a flow rate between five microliters per minute and one milliliter per minute. At box 410, the method 400 includes allowing diffusion through the removable membrane. The method 400 may also include culturing at least one of the first kind of cells, the second kind of cells, and the third kind of cells over a period of time. Additionally, the method 400 may include removing the membrane from the apparatus and retrieving at least one of the first kind of cells and the second kind of cells.

While various examples herein are described in reference to microfluidic structures, any use of microfluidic structures herein would apply to microfluidic structures as well. Therefore, the techniques described herein should be understood to apply to both microfluidic and macrofluidic domains. Fluidic microdevices as described in the foregoing may be adapted for use in either domain. As used herein, reference to microfluidic structures, microfluidic channels, etc. refers to devices having a small scale (such as a micron scale) in size and/or to devices that operate on small volume of liquid (μL, nL, pL, or fL), while macrofluidic structures, channels, etc., refers to a scale larger than microns in size and/or devices that operate a volume of liquid larger than μL.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

What is claimed:

1. An apparatus comprising:
a first plate having an upper surface, a lower surface, a proximal end, and a distal end;
a second plate having an upper surface, a proximal end, and a distal end, the upper surface of the second plate configured for placement adjacent the lower surface of the first plate;
a first media channel disposed in the first plate, the first media channel having a flow direction from the proximal end to the distal end of the first plate, the first media channel comprising
a first media inlet in the upper surface of the first plate,
an upper compartment downstream of first media inlet in the flow direction,
a removable membrane downstream of the first media inlet in the flow direction and provided in a passageway extending through the lower surface of the first plate, and
a first media outlet in the upper surface of the first plate downstream of the removable membrane in the flow direction; and
a second media channel disposed in the second plate, the second media channel having a flow direction from the proximal end to the distal end of the second plate, the second media channel comprising
a second media inlet in the upper surface of the second plate,
a lower compartment extending through the upper surface of the second plate downstream of the second media inlet in the flow direction and configured for placement adjacent passageway of the removable membrane of the first media channel, and
a second media outlet in the upper surface of the second plate downstream of the lower compartment.

2. The apparatus of claim 1, comprising two first media channels and two second media channels.

3. The apparatus of claim 1, wherein the first plate further comprises a second media inlet aperture and a second media outlet aperture, the second media inlet aperture configured for placement adjacent the second media inlet of the second plate, and the second media outlet aperture configured for placement adjacent the second media outlet of the second plate.

4. The apparatus of claim 1, further comprising a cap having a lower surface, a first media inlet aperture, and a first media outlet aperture, the lower surface of the cap configured for placement adjacent the upper surface of the first plate, the first media inlet aperture configured for placement adjacent the first media inlet of the first plate, the first media outlet aperture configured for placement adjacent the first media outlet of the first plate.

5. The apparatus of claim 4, wherein the lower surface of the cap covers the first media channel from the first media inlet to the first media outlet.

6. The apparatus of claim 4, wherein the first plate includes a detent, and the cap includes an extension that is complementary to the detent and configured for placement in the detent.

7. The apparatus of claim 1, wherein the upper compartment is populated with a first type of cells, and the removable membrane includes a second type of cells on a first side and a third type of cells on a second side.

8. The apparatus of claim 1, further including a first media that is circulated through the first media channel, and a second media that is circulated through the second media channel.

9. The apparatus of claim 7, wherein the first media includes media conditioned by a first type of cells and the second media includes a fourth type of cells.

10. The apparatus of claim 8, wherein the removable membrane is exposed to the media conditioned by the first type of cells and allows cytokine diffusion through the removable membrane and into the second media in the second media channel.

11. An apparatus comprising:
a plate having an upper surface, a lower surface, a proximal end, a distal end, and an intermediate plane;
a first media channel disposed in the plate at or above the intermediate plane, the first media channel having a flow direction from the proximal end to the distal end of the plate, the first media channel comprising
a first media inlet in the upper surface of the plate,
a removable membrane downstream of the first media inlet in the flow direction and provided in a passageway extending through the intermediate plane, and
a first media outlet in the upper surface of the plate downstream of the removable membrane in the flow direction; and
a second media channel disposed in the plate, the second media channel having a flow direction from the proximal end to the distal end of the plate, the second media channel comprising
a second media inlet in the upper surface of the plate,
a lower pathway disposed below the intermediate plane downstream of the second media inlet,
a compartment fluidly connected to the lower pathway and further connected to the passageway of the removable membrane of the first media channel, and
a second media outlet in the upper surface of the plate downstream of the compartment.

12. The apparatus of claim 11, further comprising a cap having a lower surface, a first media inlet aperture, a first media outlet aperture, a second media inlet aperture, and a second media outlet aperture, the lower surface of the cap configured for placement adjacent the upper surface of the plate, the first media inlet aperture configured for placement adjacent the first media inlet, the first media outlet aperture configured for placement adjacent the first media outlet, the second media inlet aperture configured for placement adjacent the second media inlet, the second media outlet aperture configured for placement adjacent the second media outlet.

13. The apparatus of claim 3, wherein the lower surface of the cap covers the first media channel from the first media inlet to the first media outlet.

14. The apparatus of claim 11, wherein the removable membrane includes one type of cells on a first side and another type of cells on a second side.

15. The apparatus of claim 11, further including a first media that is circulated through the first media channel, and a second media that is circulated through the second media channel.

16. The apparatus of claim 15, wherein the first media includes media conditioned by one type of cells and the second media includes another type of cells.

17. The apparatus of claim 16, wherein the removable membrane is exposed to the first media and allows cytokine diffusion through the removable membrane and into the second media in the second media channel.

18. An apparatus comprising:
a well having a first media receptacle and an open bottom;
a first plate having an upper surface, a lower surface, a proximal end, and a distal end, the upper surface of the first plate configured for placement adjacent the open bottom the well, the lower surface of the first plate including a recessed trench;
a removable membrane configured for placement in a passageway between the open bottom of the well and the upper surface of the first plate;
a second plate having an upper surface, a proximal end, and a distal end, the upper surface of the second plate configured for placement adjacent the lower surface of the first plate with the proximal ends and distal ends of the first plate and the second plate aligned, the upper surface of the second plate including a raised trough;
a second media channel formed between the recessed trench of the first plate and the raised trough of the second plate, the second media channel having a flow direction from the proximal ends of the first plate and the second plate to the distal ends of the first plate and the second plate, the second media channel comprising
a second media inlet aperture through the first plate,
a compartment through the first plate downstream of the second media inlet aperture in the flow direction and configured for placement adjacent the passageway of the removable membrane;
a second media outlet through the first plate.

19. The apparatus of claim 18, comprising two wells, two removable membranes, two passageways, and two second media channels, each well having a first media receptacle.

20. The apparatus of claim 18, wherein the removable membrane includes one type of cells on a first side and another type of cells on a second side.

21. The apparatus of claim 18, further including a first media that is contained in the first media receptacle, and a second media that is circulated through the second media channel.

22. The apparatus of claim 21, wherein the first media includes media conditioned by one type of cells and the second media includes another type of cells.

23. The apparatus of claim 22, wherein the removable membrane is exposed to the first media and allows cytokine diffusion through the removable membrane and into the second media in the second media channel.

24. A method of creating a microenvironment, the method comprising:
supplying an apparatus comprising a first media location, a second media channel, and a removable membrane between the first media location and the second media channel;
populating the removable membrane with a first kind of cells and a second kind of cells;
providing a first media in the first media location;
flowing a second media having a third kind of cells through the second media channel;
allowing diffusion through the removable membrane.

25. The method of claim 24, further comprising culturing at least one of the first kind of cells, the second kind of cells, and the third kind of cells over a period of time.

26. The method of claim 24, wherein the second media flows through the second media channel at a flow rate between five microliters per minute and one milliliter per minute.

27. The method of claim 24, wherein the first media location is a first media channel, and wherein providing a first media in the first media location includes flowing the first media through the first media channel.

28. The method of claim 24, wherein the first media location is a well having a first media receptacle and an open bottom.
